(19) United States
(12) Patent Application Publication
(10) Pub. No.: US 2010/0021904 A1
(43) Pub. Date: Jan. 28, 2010

(54) SHIELDED CROSS-LINKING PROBES

(76) Inventors: Niles A. Pierce, Pasadena, CA (US); Peng Yin, Pasadena, CA (US); Jeff Vieregg, Studio City, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/467,755

(22) Filed: May 18, 2009

Related U.S. Application Data
(60) Provisional application No. 61/128,411, filed on May 21, 2008.

(51) Int. Cl. C12Q 1/68; C07H 21/04; C07H 1/00; G01N 33/00; C12P 19/34

(52) U.S. Cl. 435/6; 536/24.3; 536/25.3; 436/94; 435/91.1

(57) ABSTRACT

The present invention relates to the use of nucleic acid probes to bind to targets. In some embodiments, the probe comprises a shielded cross-linking probe.

### CROSS-REFERENCE TO RELATED APPLICATIONS

The present application claims priority to U.S. Provisional Application No. 61/128,411, filed May 21, 2008, which is hereby incorporated by reference in its entirety.

### SEQUENCE LISTING

The present application is being filed along with a Sequence Listing in electronic format. The Sequence Listing is provided as a file entitled Seq_Listing_CALTE-049A, created May 18, 2009, which is 688 bytes in size. The information in the electronic format of the Sequence Listing is incorporated herein by reference in its entirety.

### STATEMENT REGARDING FEDERALLY SPONSORED R&D

This invention was made with government support under grant nos. NIH 5R01 EB006192-04 “Hybridization chain reaction: in situ amplification for biological imaging” and NIH P50 HG004071 “Center for in toto genomic analysis of vertebrate development”.

### BACKGROUND

1. Field

The present disclosure relates to various probes that can bind and cross-link to a target nucleic acid.

2. Background

Molecules that selectively bind to nucleic acids have a large variety of uses, including modifying gene regulation and/or protein expression, as well as being able to serve as indicators for the presence or absence of a specific nucleic acid in a sample. One example of an indicator role is in the technique of in situ hybridization, which allows for the spatial mapping of nucleic acid sequences, such as mRNAs, in normal and pathological tissues. In situ hybridization can be used to study gene expression and regulation in a morphological context from the sub-cellular to the organismal levels (Lawrence, J. B., R. H. Singer, and L. M. Marselle, Highly localized tracks of specific transcripts within interphase nuclei visualized by in situ hybridization. Cell, 1989. 57: p. 493-502; Kislauskis, E. H., et al., Isoform-specific 3′-unstranslated sequences sort alpha-cardiac and beta-cytoplasmic actin messenger RNAs to different cytoplasmic compartments. The Journal of Cell Biology, 1993. 123(1): p. 165-172; Wilkie, G. S., et al., Transcribed genes are localized according to chromosomal position within polarized Drosophila embryonic nuclei. Current Biology, 1999. 9: p. 1263-1266; Levsky, J. M., et al., Single-cell gene expression profiling. Science, 2002. 297: p. 836-840; Qian, X. and R. V. Lloyd, Recent developments in signal amplification methods for in situ hybridization. Diagnostic Molecular Pathology, 2003. 12(1): p. 1-13; Qian, X., L. Jin, and R. V. Lloyd, In situ hybridization: basic approaches and recent development. The Journal of Histotechnology, 2004. 27(1): p. 53-67; Kosman, D., et al., Multiplex detection of RNA expression in Drosophila embryos. Science, 2004. 305: p. 846.)

### SUMMARY OF THE INVENTION

In some aspects, probes and/or methods involving a molecular conformational change is/are used to provide an enhanced level of specificity for the formation of cross-links between a probe and its target. In some embodiments, these cross-links then allow for the use of ultra-stringent washing to eliminate the remaining probes from the sample.

In some aspect, a new approach to in situ hybridization is provided. It achieves high specificity using methods that, in some embodiments, are suitable for sensitive, multiplexed, quantitative bioimaging in fixed cells, tissue sections, and whole-mount embryos.

In some aspects, probes and/or methods involving the selective displacement of a blocking region from a probe that contains a cross-linker are provided. The displacement of the blocking region can allow for superior selectivity in creating a cross-link between a probe and a target nucleic acid.

In some aspects, a cross-linking probe is provided that comprises an initiator region; a probe region, wherein the probe region is linked to the initiator region; at least one cross-linker that is part of the probe region; and a blocking region that is hybridized to the probe region such that the blocking region reduces the cross-linking of the cross-linker to other molecules when the blocking region is hybridized to the probe region.

In some aspects, a method of associating a cross-linking probe with a nucleic acid sequence is provided that comprises providing a cross-linking probe and a nucleic acid sequence; wherein the cross-linking probe comprises: an initiator region; a probe region, wherein the probe region is linked to the initiator region; at least one cross-linker that is part of the probe region; and a blocking region hybridized to the probe region. The method can further comprise hybridizing the initiator region to a part of the nucleic acid sequence; dissociating the blocking region from the probe region; hybridizing the probe region to a second part of the nucleic acid sequence; and cross-linking the cross-linker.

In some aspects, a method of determining the presence or absence of a nucleic acid is provided and comprises providing a sample; adding to the sample an initiator region that is linked to a probe region, wherein there is at least one uncross-linked cross-linker that is part of the probe region, and wherein a blocking region is hybridized to the probe region when the initiator region is added. The method can further comprise hybridizing the initiator region to a first part of a nucleic acid contained within the sample, if the nucleic acid is present in the sample; dissociating the blocking region from the probe region, if the nucleic acid is present in the sample; hybridizing the probe region to a second part of the nucleic acid, if the nucleic acid is present in the sample; cross-linking the cross-linker; performing a wash following the cross-linking; associating the probe region with a detectable marker; and detecting the presence or absence of the detectable marker, thereby determining the presence or absence of a nucleic acid.

### BRIEF DESCRIPTION OF THE DRAWINGS

FIG. 1A depicts some embodiments of a cross-linking probe.

FIG. 1B is a flow chart outlining some embodiments of using a cross-linking probe.

FIG. 2 depicts some embodiments of using a cross-linking probe.

FIG. 3 depicts an embodiment of making a cross-linking probe.

FIG. 4 depicts some embodiments of a cross-linking probe.

FIG. 5 depicts some embodiments of a cross-linking probe.

FIG. 6 depicts an embodiment of an HCR method employing a cross-linking probe.

FIG. 7 depicts two different photoactivatable crosslinkers and the gel results from Example 5.

### DETAILED DESCRIPTION OF THE PREFERRED EMBODIMENT

The present disclosure provides a wide variety of alternative cross-linking probes and various methods of using them. In some embodiments, these probes and techniques provide the ability to create covalent bonds to their target nucleic acid and to do so with a high degree of selectivity and/or specificity. In some embodiments, these probes and techniques allow for the selective cross-linking of the probe to a specific nucleic acid. In some embodiments, the cross-linking itself is selective in that cross-linking between unbound cross-linking probes and non-target (and non-probe) nucleic acids is significantly reduced or prevented.

In some embodiments, a shielded cross-linking probe includes a probe region, a cross-linker (that is in or part of the probe region) and a blocking region that reduces the ability of the cross-linker to cross-link and/or the probe region to bind to undesired sequences. The blocking region will dissociate from the probe region in the presence of the target nucleic acid, which then allows the cross-linker to cross-link to the desired target. In some embodiments, the selectivity of the cross-linker is enhanced further by the use of an activatable cross-linker, which allows one to determine when and/or where in a reaction, sample, or system the cross-linker should become capable of cross-linking. The above selective cross-linking can provide a variety of useful functions. For example, the specifically cross-linked probe can be used in an in situ hybridization technique which can then employ ultrastringent washing conditions in order to obtain superior in situ results. Of course, any process in which very specific and very tight (e.g., covalent level strength) binding between a molecule and a target nucleic acid are beneficial can benefit from one or more of the embodiments disclosed herein.

In some embodiments, shielded cross-linking probes will form a crosslink to a target (such as mRNA) with high specificity by employing one or more of the following: 1) initial sequestration of one or more activatable cross-linkers within a duplex portion of a nucleic acid probe; 2) stringent sequence filtering via competitive branch migration replacement of blocker/probe region base pairs with probe/target base pairs such that upon completion of the triggered conformation change, the cross-linkers are sequestered within a new probe/target duplex; and 3) high-yield activation of the cross-linkers. These properties, especially when more than one is present, will ensure that the probe covalently cross-links to the sample primarily (and in some embodiments only) when it is specifically base-paired to its complementary target.

The following section outlines the definitions of some of the terms used herein as well as providing some alternative embodiments. Following that section, a general description of how the cross-linking probes can be used and the various components is provided. Following that section, a description of various alternative embodiments, which also includes a description of variations of the various elements disclosed herein, is provided. Following that section, a series of Examples outlining some possible uses of some of the disclosed embodiments is provided.

### DEFINITIONS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual

(Third ed., Cold Spring Harbor Laboratory Press, Cold Spring Harbor, N.Y. 2000). The nomenclatures utilized in connection with, and the laboratory procedures and techniques of described herein are those well known and commonly used in the art.

As utilized in accordance with the embodiments provided herein, the following terms, unless otherwise indicated, shall be understood to have the following meanings:

The term “nucleic acid” refers to natural nucleic acids, artificial nucleic acids, non-natural nucleic acid, orthogonal nucleotides, analogs thereof, or combinations thereof. Nucleic acids may also include analogs of DNA or RNA having modifications to either the bases or the backbone. For example, nucleic acid, as used herein, includes the use of peptide nucleic acids (PNA). The term “nucleic acids” also includes chimeric molecules.

As used herein, the terms “polynucleotide,” “oligonucleotide,” and “nucleic acid oligomers” are used interchangeably and mean single-stranded and double-stranded polymers of nucleic acids, including, but not limited to, 2′-deoxyribonucleotides (nucleic acid) and ribonucleotides (RNA) linked by internucleotide phosphodiester bond linkages, e.g. 3′-5′ and 2′-5′, inverted linkages, e.g. 3′-3′ and 5′-5′, branched structures, or analog nucleic acids. Polynucleotides have associated counter ions, such as H⁺, NH₄⁺, trialkylammonium, Mg²⁺, Na⁺ and the like. A polynucleotide can be composed entirely of deoxyribonucleotides, entirely of ribonucleotides, or chimeric mixtures thereof. Polynucleotides can be comprised of nucleobase and sugar analogs. Polynucleotides typically range in size from a few monomeric units, e.g. 5-40 when they are more commonly frequently referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytidine, “G” denotes deoxyguanosine, and “T” denotes thymidine.

The term “hairpin” refers to a structure formed by intramolecular base pairing in a single-stranded polynucleotide ending in an unpaired loop. A “hairpin loop” refers to a single stranded region that loops back on itself and is closed by a single base pair.

The term “initiator region” denotes a section of a probe that can initially bind to a target nucleic acid, or at least a part of the target nucleic acid. In some embodiments, the initiator region is generally two to 1000 nucleotides, such as 3-100 nucleotides or 4-50 nucleotides. In some embodiments, there can be more than one initiator. For example, in some embodiments there can be two initiators, one on each side of the probe region.

The term “probe region” denotes a section of a hybridization probe that binds to a target nucleic acid. In some embodiments, this is distinguishable from the initiator region in that an initiator region, if present, will bind to the target first. This initial binding allows for the initiation of the displacement of the blocking region via a branch migration process. In some embodiments the probe region is 3-1000 nucleotides in length, such as 6-100 or 10-50 nucleotides in length.

The term “linked” or “links” denotes that two regions are covalently connected to one another. There can be additional intervening structures between the two regions. Thus, the linking can be direct (also described as being “immediately adjacent” ) or indirect. While two nucleic acids that become cross-linked could be characterized as “linked,” for the sake of clarity, such links are referred to herein as “cross-links” and not generally denoted by the term “link”. Thus, a single nucleic acid that includes a probe region, a cross-linker, a loop region, and a blocking region would all be “linked” to each other, and if the cross-linker cross-linked to the blocking region, then the cross-linker (and the probe region that the cross-linker is in) would be “cross-linked” to the blocking region.

The term “associated” denotes that the relevant structures and/or regions are localized with one another by some type of binding interaction. Association can be due to covalent bonds (e.g., linked or cross-linked) or they can be due to noncovalent bonds (e.g., hybridization, antibody binding, etc.)

The term “cross-linked” denotes that a cross-linker has formed a covalent bond with another residue, molecule, nucleotide, etc. The bond can be formed within the same molecule (e.g., cross-linking a hairpin loop shut) or can be between molecules (e.g., cross-linking a cross-linking probe to a target molecule). “Cross-linked” can include one or more cross-linked bonds.

The term “cross-linker” denotes a molecule or atom that is capable of forming a covalent bond to another molecule. While the cross-linkers should allow for two separate molecules (e.g., nucleic acids) to be effectively cross-linked together, more than one cross-linker can be used, thus, any single cross-linker does not need to be strong enough to keep the two molecules bonded together. In some embodiments, the cross-linker is capable of forming a covalent bond between the target nucleic acid and a probe region.

The term “blocking region” denotes a structure that obstructs the probe region and cross-linker when it is hybridized to the probe region. While the blocking region need not completely prevent any interaction of the cross-linker and/or probe region with the environment, it will significantly reduce this interaction so as to allow a greater degree of specificity of cross-linking that will depend upon the presence or absence of the blocking region. The blocking region is displaced via a branch migration in which base-pairs between the probe region and blocking region are replaced one at a time by base pairs between the probe region and the target. Hence, the cross-linkers are shielded (and in some embodiments are always effectively shielded) from the biological sample either within the probe or between the probe and the intended target. In some embodiments, the blocking region contains one or more areas that are inert to cross-linking. For example, the blocking region can contain a subregion that is just the polymer backbone without any bases. The blocking region can be a single strand of nucleotides or one or more strands of nucleotides.

The term “reversibly hybridize” denotes that the molecule can hybridize and dissociate (or be displaced from) from a nucleic acid. Nucleic acids that are cross-linked to one another will not dissociate due to the covalent interaction. However, molecules that include cross-linkers that have not been activated can still reversibly hybridize or dissociate from a complementary molecule. In addition, a molecule can be described as being “reversibly hybridizable,” even if it has a cross-linker in it, as long as it can dissociate from a binding molecule.

The term “reduce” denotes some decrease in amount. In some embodiments, an event is reduced by 1, 2, 3, 4, 5, 10, 15, 20, 25, 30, 35, 40, 45, 50, 55, 60, 65, 70, 75, 80, 85, 90, 95, 96, 97, 98, 99, 99.9, 99.99, 99.999, percent or more, including any value above any of the preceding values, as well as any range defined between any two of the preceding values.

The term “loop region” denotes a region that link a probe region and a blocking region. This region is optional. The loop region can include nucleotides and/or simply be a polymer backbone without any bases.

The modifier “sub” denotes a component of a region.

The term “detectable marker” denotes any molecules that can be observed and/or detected, either directly (e.g., fluorescence), or indirectly (e.g., detection of a product, where the presence of the product is based upon the presence of a target). For indirect detectable markers, the molecule that acts as the intermediate can be the “detectable marker,” even if it is a product from that molecule that is actually detected. In some embodiments, a detectable marker is a part of the probe that can serve as a marker, such as in a surface plasmon resonance technique (such as in a BIACORE™ machine). Thus, in some embodiments, no separate detectable marker need be included.

The term “amplifier molecule” denotes a molecule that allows for the association of a detectable marker with a probe region. This association can be indirect, e.g., through an additional pairing region. The amplifier can be cross-linked to the cross-linking probe, but need not be.

The term “pairing region” denotes a nucleic acid sequence that can hybridize to a complementary pairing region.

The term “complementary pairing region” denotes a nucleic acid sequence that can hybridize to a pairing region.

The term “orthogonal nucleotides” denotes those nucleotides that will not effectively basepair with a natural nucleotide. Natural nucleotides are defined as adenine, cytosine, guanine, thymine, and uracil. Some types of orthogonal nucleotides contain modified bases, such as isoC and isoG.

The term “external linker” denotes one or more atoms that serve to link two molecules together. A cross-linker can be attached to or include an external linker, which can allow for greater flexibility in the positioning of the cross-linker. The term “external” is used to further distinguish this linking aspect from a cross-linker (which is capable of forming a cross-link), as an external linker need not be capable of actually forming a cross-link after it has been connected and can merely serve as a longer tether to attach, for example, the cross-linker to the probe region.

The term “activated” denotes that the cross-linker has been exposed to a stimulus that promotes cross-linking.

The term “activatable” denotes that a cross-linker has a state dependent ability to cross-link. In some embodiments, the “state” is an environmental factor, such as radiation (UV, visible, etc.), presence of a particular chemical compound, or a conformational change (such as the presence of absence of the blocker region).

The term “unactivated” denotes that an activatable cross-linker has not been activated, and thus, the likelihood that a cross-link will be formed is very low.

The term “un-cross-linked” denotes that the cross-linker has not been cross-linked. A molecule can be activated and still be uncross-linked.

The term “ultrastringent wash” denotes a washing condition that is much more effective at weakening hybridization interactions than those commonly used for in situ hybridization. Examples include elevated temperatures and high concentrations of chemical denaturants such as formamide, organic cosolvents, or urea. In some embodiments, an ultrastringent wash would completely eliminate nucleic acid hybridization in the sample, thus causing any non-crosslinked probes to dissociate from their binding sites. In some embodiments, an ultrastingent wash would typically be one that is strong enough to destabilize all base-pairing (including orthogonal base pairs) between the probe and the sample. This could be achieved using higher temperatures or (e.g. high concentration of formamide). In some embodiments, an ultrastringent wash comprises 2×SSC and 75% formamide at 70 degrees. In some embodiments, 20-50% formamide is employed instead of 75%. In some embodiments, these wash conditions are for relatively short probes of less than 1000 nucleotides, for example, 900, 800, 700, 600, 500, 400, 300, 200, 100, 50, 40, 30, 20, 10 or fewer nucleotides, including any range defined between any two of the preceding values.

The term “target nucleic acid” denotes a nucleic acid of interest. In some embodiments, the target nucleic acid is one that a user wishes to determine the presence or absence of. In some embodiments, the target nucleic acid is one that the user may wish to specifically attach a cross-linker to. Exemplary target nucleic acids include DNA, RNA, mRNA, and miRNA.

The term “cross-linking probe” denotes a molecule that is capable of selectively binding to a target nucleic acid and then cross-linking itself to that nucleic acid. While cross-linking probes can be used to “probe” for specific target nucleic acids in a sample, a “cross-linking probe” is not limited to this use and need not include a detectable marker or other detectable aspect. For example, a cross-linking probe can include only a nucleic acid probe region and a cross-linker. Such a probe can be used to obstruct or inactivate a target nucleic acid, rather than for the detection of the presence or absence of the nucleic acid. Of course, the probes can include the other aspects disclosed herein as well.

### Cross-Linking Probes

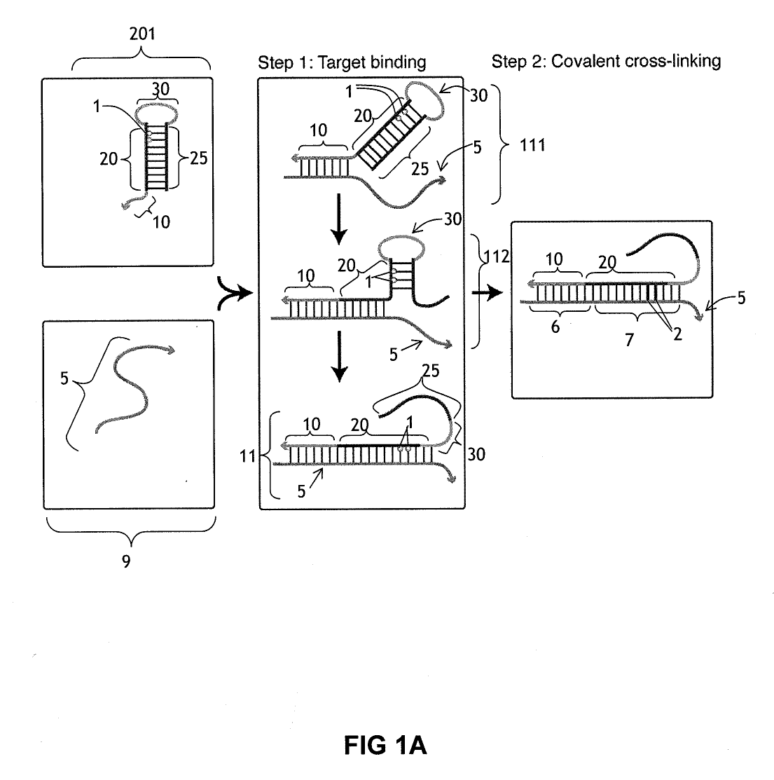

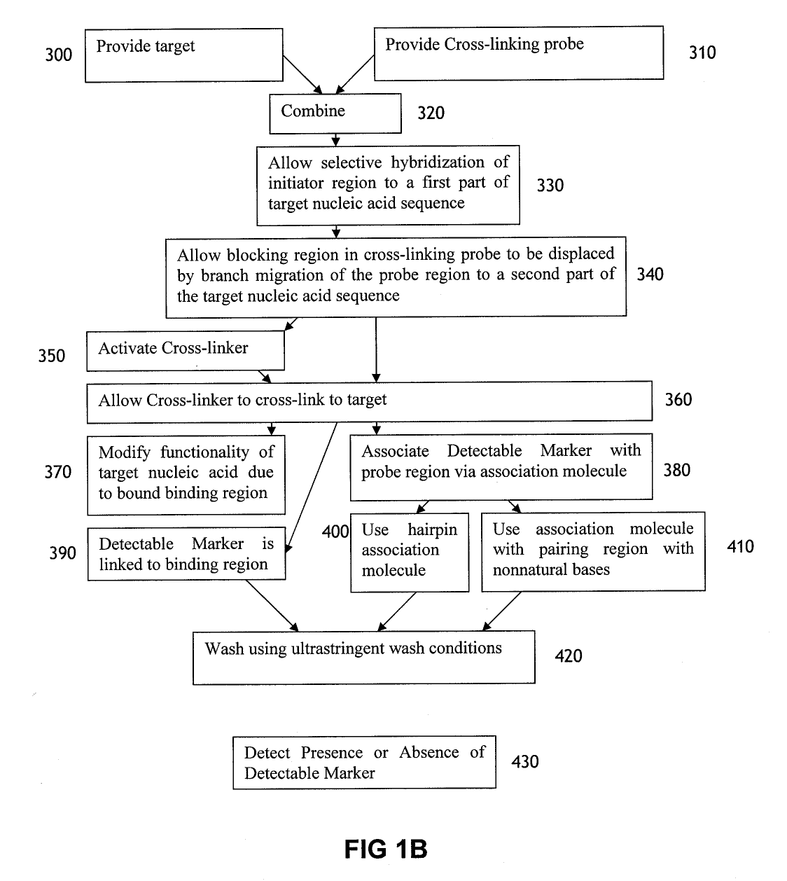

The upper-left corner of FIG. 1A depicts an embodiment of a cross-linking probe. This cross-linking probe 201 comprises a single-stranded initiator region 10 that is linked to a probe region 20, which includes at least one cross-linker 1. Initially, cross-linker 1 and/or probe region 20 are/is effectively obstructed from freely interacting with the environment (in particular, any part of the sample that is not the intended target sequence) by a blocking region 25 that is effectively complementary to the probe region 20 (complementary enough to allow this beneficial blocking to occur). In the depicted embodiments, there is an optional loop region 30, which links the probe region 20 to the blocking region 25. As shown in FIG. 1A, combining a target nucleic acid 5 with the cross-linking probe 201 allows for the initiator region 10 to hybridize to the target sequence (depicted as process 111), which allows for branch migration up the cross-linking probe (depicted as process 112) and thus for the separation of the probe region 20 from the blocking region 25. This then allows for the base-pairing of the initiator region 10 and the probe region 20 to the target nucleic acid 5 (depicted as process 11). Following this, in some embodiments, one can then activate the cross-linker(s) 1 via photo-activation or from the conformational change, to allow the cross-linker(s) 1 to form cross-link(s) 2 between the probe region 20 and the target nucleic acid 5 (in particular, the section of the target that is complementary to the probe region 7). In some embodiments, the initiator region is immediately adjacent to the probe region so as to promote effective strand displacement of the blocking region 25 upon binding of the initiator region 10 to target.

As noted above, a specific and tight association between a target and another molecule can be beneficial in any technique where a relatively high degree of specificity and tight association is beneficial. FIG. 1B provides a flow chart outlining a variety of possible steps in various methods of using such a cross-linking probe. As shown in FIG. 1B, one can start by providing (e.g., making and/or obtaining) a target nucleic acid 300 (or a sample that is suspected of having a target nucleic acid), providing the cross-linking probe 310, and combining the two 320. One then allows (and/or promotes) the selective hybridization of the initiator region to at least a part of the target nucleic acid 330 and allows (and/or promotes) the selective hybridization of the probe region to a second part of the target nucleic acid sequence 340. In embodiments that employ an activatable cross-linker, one can then activate the cross-linker 350. One then allows the cross-linker to cross-link to the target 360. In some embodiments, this can achieve the result of modifying the functionality of the target nucleic acid 370. In some embodiments, e.g., when the probe is used for in situ type methods, it can be useful to have detectable markers associated with the probe region. In some embodiments, the detectable marker (and/or the detectable marker region) is already part of the cross-linking probe and can be linked, directly or indirectly to the probe region 390. In some embodiments, the detectable marker region is associated with the probe region via an amplifier molecule, in process 380. This can be achieved via a pairing region and a sequence that is complementary to the pairing region. In some embodiments, a hairpin structure is used as the amplifier molecule 400. In some embodiments, the pairing region and pairing region complement are paired via the use of orthogonal bases that do not hybridize with natural nucleic acids. 410. Once one or more detectable markers are associated with the probe region, the sample can be washed to remove any non-cross-linked probes. In some embodiments, given that the desired probe is cross-linked to the desired target, ultrastringent wash conditions can be used 420. Following the wash, one can examine the sample for the presence or absence of the detectable marker to determine whether or not the target nucleic acid is present in the sample.

While the above sections describe some various general embodiments, the following section provides more detailed specific embodiments of using the cross-linking probes.

### Additional Cross-Linking Probe Embodiments

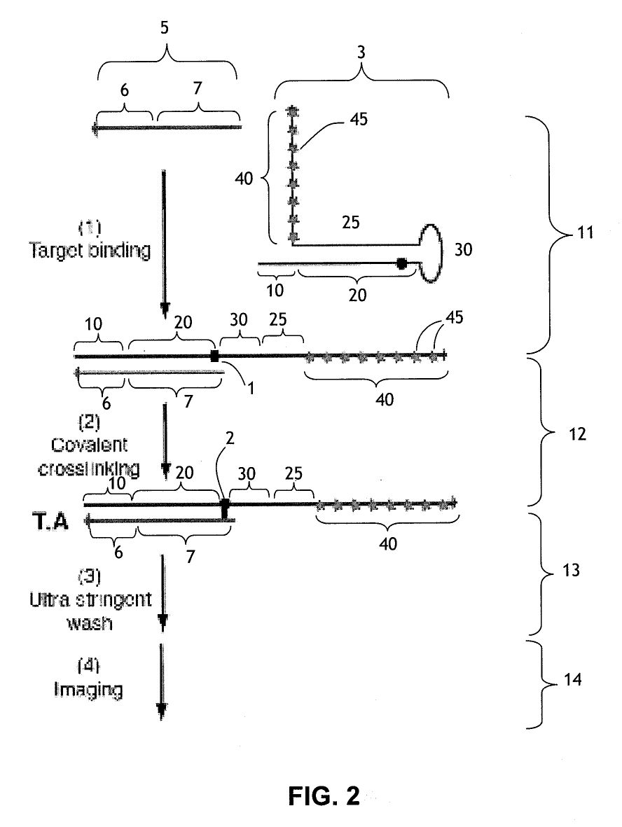

FIG. 2 depicts an embodiment of a cross-linking probe and its method of use. Initially, one binds the target nucleic acid with a cross-linking probe 3 (in process 11). The probe molecule 3 is introduced to the sample containing the target nucleic acid. The exposed initiator region 10 base-pairs to the target 5 at a first region 6, allowing rapid nucleation with the target (a kinetic effect) and providing affinity for the target via the formation of new base-pairs (a thermodynamic effect). After the probe 3 nucleates with the target mRNA via base-pairing at a first region 6, the target 5 base-pairs to the probe region 20 via a branch migration that opens a hairpin loop (which is an optional structure). The opening of the hairpin loop provides an entropic benefit that increases the strength of the interaction between the probe 3 and the target 5 (as would the dissociation of the blocking region if there is no loop). In some embodiments, the bases within the probe region carry activatable cross-linkers 1, which are now base-paired to the target 5. The blocking region 25 can provide a stringent specificity check that helps ensure that the bases carrying activatable cross-linkers 1 primarily (and, in some embodiments, only) pair to endogenous nucleic acids if they are specifically paired to the target 5. While not intending to be limited to theory, it is understood that this is because it is energetically prohibitive to open the stem to expose the cross-linkable bases 1 except via a branch migration process in which the intra-stem base pairs are replaced one-by-one by intermolecular base-pairs between the probe 3 and the target 5. The presence of each additional base pair in the stem between the initiator region 10 and the cross-linkable base(s) 1 increases the specificity stringency.

Next, one can covalently cross-link the probe region to the target in process 12. In some embodiments, one allows sufficient time for the probe molecules to diffuse into the sample and bind to target mRNAs. Following this, the covalent cross-linkers 1 are photo-activated leading to probe-target covalent cross-linking, which includes at least one cross-link 2. In some embodiments, only those probes base-paired specifically to target nucleic acids are covalently linked to the sample. In some embodiments, all other probes become fused in the closed state due to covalent linking of the protection and propagation regions. As discussed herein, there are a variety of mechanisms for activating the cross-linking process, if one is employing an activatable cross-linker.

Once the specifically bound probes are covalently linked to the sample, it is possible to employ one or more ultra-stringent washes (process 13) that remove a significant amount of all other probes from the sample to yield exquisite specificity. In some embodiments, more of the other probes can be removed than would be typical from traditional washes. In some embodiments, all (or substantially all) of the non-cross-linked cross-linkable probes are removed. This can include any probes that are not hybridized to the sample, that are partially hybridized to the sample, or that are hybridized but not cross-linked to the sample. In some embodiments, all probes that are not crossed linked to the target nucleic acid are removed. In some embodiments, substantially all of the uncrosslinked probe is removed. In some embodiments, substantially all of the detectable probe that has not been cross-linked to a non-probe nucleic acid is removed. In some embodiments, substantially all of the detectable probe that has not been cross-linked to a non-probe nucleic acid is removed. In some embodiments, the only unbound probe that remains after the wash is readily identifiable as being background. Once the specifically bound probes are cross-linked to their targets, it is possible to employ one or more ultra-stringent washes (process 13) to remove non-cross-linked probes. In some embodiments, substantially all non-cross-linked probes are removed from the sample. In some embodiments, 90,% or more of the non-crosslinked probes are removed, for example 91, 92, 93, 94, 95, 96, 97, 98, 99, 99.9%, 99.99% or more of the uncrosslinked probe is removed. In some embodiments, the background signal per cell is less than the strength of the amplified signal for a correct target.

Once a significant amount of the non-crosslinked probe has been washed away, the sample can be imaged 14. In some embodiments, fluorescence microscopy can be employed to image the location of the cross-linked cross-linking probes, thus revealing the location of the target nucleic acids.

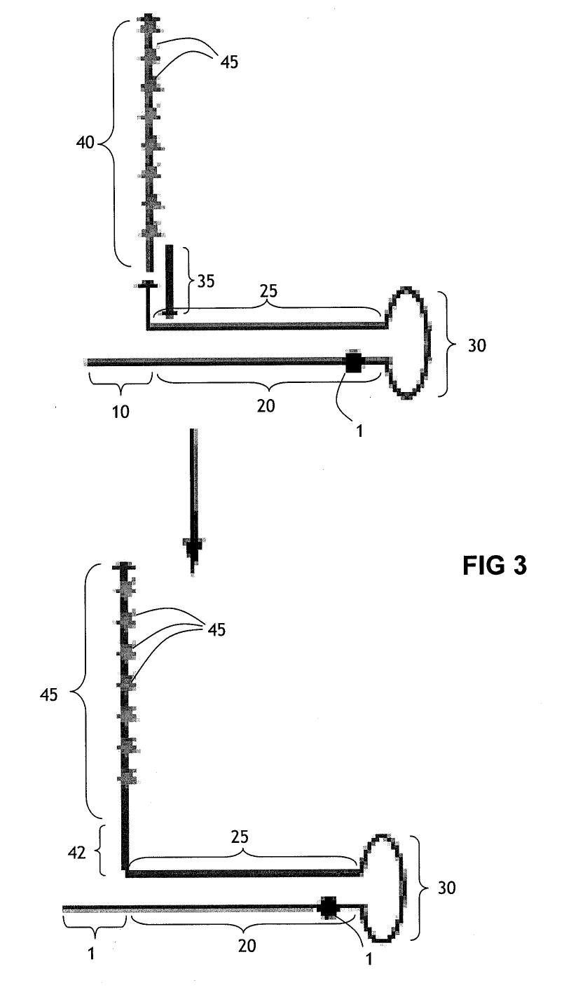

In some embodiments, the cross-linking probe, instead of carrying fluorophores on a single-stranded DNA tail, can employ branched DNA structures or other non-linear DNA structures as fluorophore carriers. In some embodiments, the detectable marker region 40 in the cross-linking probe 3 has an independent sequence from the probe region. Thus, the modular approach shown in FIG. 3 can be used for synthesizing some embodiments of the cross-linking probe. The detectable marker region 40 and the other part of the cross-linking probe (25, 30, 20, 10, and 1) are synthesized separately, and then ligated using nucleic acid 35 to link the two parts together to produce a complete cross-linking probe. In some embodiments, the detectable marker region 40 can have an independent sequence from the target, and thus, the detectable marker region can be reused for different targets.

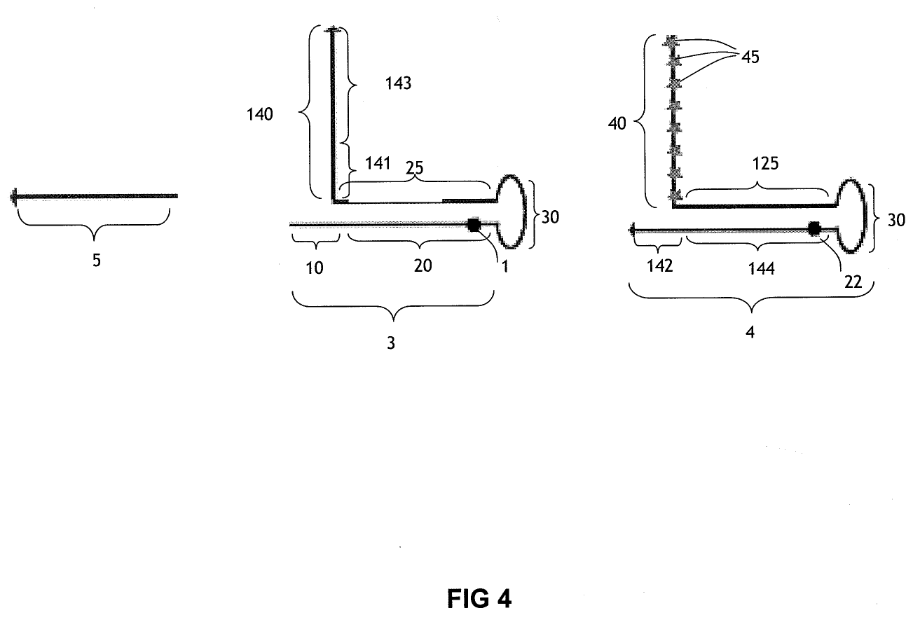

FIG. 4 depicts another embodiment of using a cross-linking probe. In this embodiment, while the probe 3, lacks a detectable marker region, the use of an amplifier molecule 4, allows for the addition of the detectable marker and/or region to be associated with the target, via the pairing region 140. In this embodiment, one allows the amplifier molecule 4 to bind to pairing region 140 via a complementary pairing region 142 and/or 144. In this embodiment, at least a part of the complementary pairing region 144 is blocked by a second blocking region 125. The amplifier molecule thus undergoes a conformational change, similar to that described for FIG. 2. One can then cross-link the amplifier molecule 4 to the cross-linked probe 3 via photo-activation of the cross-linker 22. Then one can perform an ultra-stringent wash to remove all other amplifiers from the sample. Thus, in some embodiments, multiple washes and multiple cross-linker activation steps can be performed. In some embodiments, multiple amplifier molecules can be used simultaneously such that each amplifier molecules targets the single-stranded tail of another species, with intermolecular covalent linkages formed only in the case of specific interactions. In some embodiments, those amplification polymers that are covalently linked to the target nucleic acid would be retained during the ultra-stringent wash.

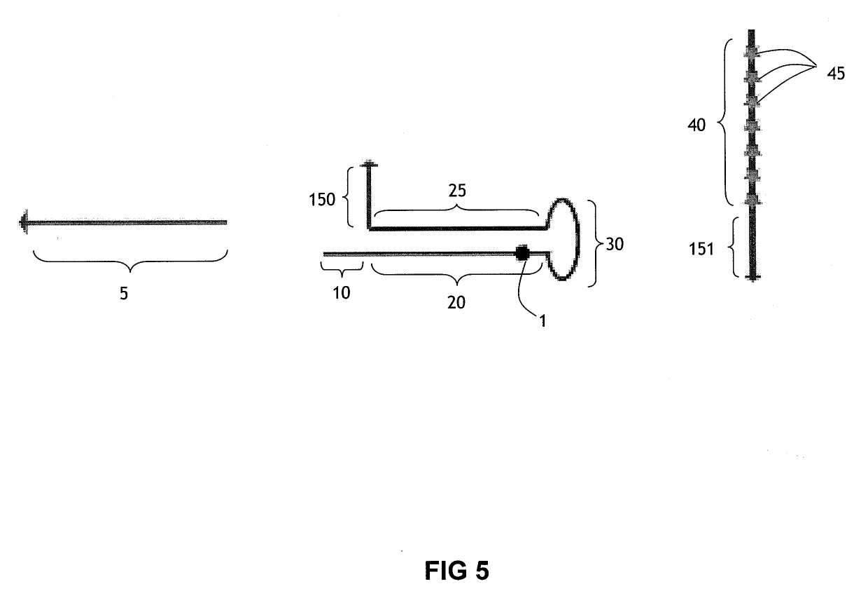

FIG. 5 depicts an embodiment which employs orthogonal base pairing (between pairing region 150 and a complementary pairing region 151) to replace the role of conformation change during amplification in the example above. In some embodiments, the pairing region 150 has no natural bases appear consecutively in the pairing region of the amplifier strand 151 (for example natural bases could alternate with orthogonal bases such as iso-C and iso-G). In some embodiments, any orthogonal base combination that will avoid, prevent, or reduce hybridization of the pairing region to native or natural nucleic acids can be used. In some embodiments, the orthogonal bases can base-pair to each other (e.g. iso-C pairs strongly with iso-G) but cannot base-pair to the natural bases (see, e.g., Collins, M. L., et al., A branched DNA signal amplification assay for quantification of nucleic acid targets below 100 molecules/ml. Nucleic Acids Research, 1997. 25(15): p. 2979-2984). As a result, the amplifier molecules cannot significantly base-pair to endogenous nucleic acids because the natural bases in the amplifier strand would be able to form only isolated base-pairs separated by energetically unfavorable interior loops (i.e. few energetically stacked base pairs could result from these non-specific interactions). By placing orthogonal bases in the pairing region 150 (and/or complement 151), one can ensure that the complementary pairing region 151 (which will contain a sequence that can hybridize to the orthogonal bases and/or contain the orthogonal bases) will base-pair primarily (and in some embodiments only) to the molecule that includes the probe region. The same approach can be used for base-pairing of multiple amplifier molecules to form a linear or branched amplification polymer in which the complementary pairing region contains sufficient orthogonal nucleotides so as to prevent any substantial or effective base pairing between the complementary pairing region and other nucleotides in the sequences. In some embodiments, the complementary pairing region 151 contains no consecutive natural bases and intermolecular base-pairing between the amplification molecules is via a combination of natural and orthogonal base pairs.

Once one has an amplifier molecule with an orthogonal complementary pairing region 151 one allows the amplifier molecule 4 to bind to pairing region 150 via a combination of natural and orthogonal base pairs. One can then cross-link the amplifier molecule 4 to the molecule containing, linked to, or associated with the probe region 20. If the cross-linker 1, requires activation, then the activation step can also be performed. Following this, one can then use an ultra-stringent wash to remove effectively all other amplifier molecules 4 from the sample.

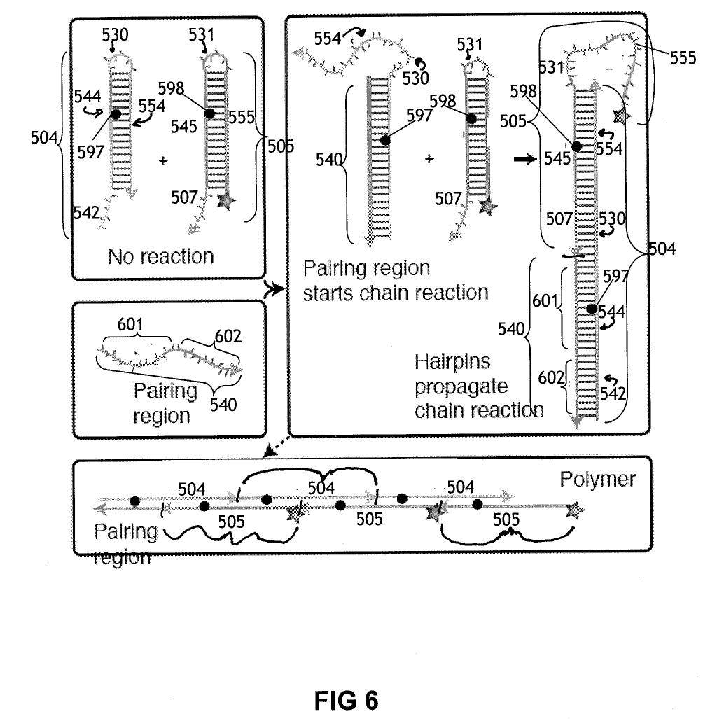

FIG. 6 depicts another embodiment in which one can employ a cross-linking probe. As shown in FIG. 6, cross-linkers can be used in other blocked probe arrangements, such as in hybridization chain reactions, as discussed in U.S. Pat. Pub. Nos. 20060228733, 20050260635, and 20060234261, the entireties of each of which, including the discussion of hairpins and their use in hybridization chain reactions, and HCR itself are incorporated herein by reference. As can be seen in FIG. 6, in one embodiment, the pairing region 540 (with subregions 601 and 602) of a probe (only the pairing region is depicted in FIG. 6, the rest of the probe (which can be a shielded cross-linking probe) can be on either side of the pairing region) can be combined with two hairpins (504 and 505) that each include a crosslinker (597 and 598). The first hairpin 504 includes an initiator region 542 (complementary to 602), a complementary pairing region 544 (complementary to 601), a loop region 530, and a blocking region 554 that is complementary to 544. The second hairpin 505 includes a second initiator region 507 that is complementary to the loop region 530, another complementary pairing region 545 that is complementary to 554, a loop region 531, which is complementary to the initiator region 542, and a blocking region 555, that is complementary to region 545 and can be the same as region 554. As shown in the figure, these monomers can be allowed to form a polymer which will include the crosslinkers (598 and 597). In turn, the polymer can then be crosslinked, resulting in a crosslinked polymer. In some embodiments, the HCR scheme will also work when some or all the polarities of the strands are reversed (arrow at the opposite ends of the probe hairpin and the amplification hairpins). The stars in FIG. 6 represent optional detectable markers.

In some embodiments, any of the described embodiments herein will also work when some or all of the polarities are reversed.

In some embodiments one can employ a shielded cross-linking probe in situations where the size of the fluorophore-carrying segment hinders penetration into the sample. In such embodiments, nucleated dendrimers (as described in P. Yin, H. M. T. Choi, C. R. Calvert, N. A. Pierce. Programming biomolecular self-assembly pathways. Nature, 451:318-322, 2008; U.S. Pat. Pub. Nos. 20090011956; and 20060234261, the entireties of each of which, including the various teachings regarding dendritic growth and HCR, are incorporated herein by reference) can be used to deliver one or more fluorophores bound to the binding probe (or a molecule associated therewith). In some embodiments, orthogonal isoC/isoG bases can be interspersed with natural bases to ensure that the dendrimer components do not base-pair non-specifically with native nucleic acids. After (or with) the assembly of the amplification dendrimer attached to the target nucleic acid/binding probe complex, the dendrimer can be covalently cross-linked to the binding probe (or molecules associated or linked thereto), and each branch of the dendrimer can be covalently linked to its parent branch. In such embodiments, the initial components of the branches can all (or some fraction thereof) include a cross-linker which can (but need not be) activatable. In some embodiments, a subsequent stringent wash can be applied to remove all other non-specifically-bound amplification molecules. In some embodiments, cross-linkers can be used in other blocked probe arrangements, such as in hairpins in hybridization chain reactions, as discussed in U.S. Pat. Pub. Nos. 20060228733 and 20060234261, the entireties of both of which, including the discussion of hairpins and their use in hybridization chain reactions, are incorporated herein by reference.

### Alternative Embodiments of Components

While, given the present disclosure, one of skill in the art will appreciate that there are a variety of alternative embodiments for each of the herein noted components, the following section briefly outlines some explicit alternative embodiments for some of these components.

As noted above, orthogonal nucleotides can be used for a variety of purposes in a cross-linking probe or the molecules that are associated therewith. In some embodiments, these orthogonal nucleotides can be used to improve or ensure specificity between an amplifier molecule and a molecule that includes a probe region. In some embodiments, they can be used to allow specificity for the parts of a dendrimer. In some embodiments, they can be used to provide for a blocking region that can block or obstruct a cross-linker, while preventing the cross-linker from forming a cross-link with the blocking region. In such an embodiment, any nucleotide that will not effectively cross-link with the cross-linker can be used. Indeed, in some embodiments, a molecule that only contains the backbone of the nucleotide and/or lacks one or more atoms involved in cross-linking (such as the bases) can be used.

The initiator region allows for the initial priming of the cross-linking probe to the target nucleic acid and can allow for subsequent strand displacement of the blocking region. This is not required for all embodiments. In some embodiments the initiator region is at least 2 and typically not more than 200, but can be as many as 1000 nucleotides or more. In some embodiments, the initiator is immediately adjacent to the probe region. In some embodiments there can be an intervening structure, as long as effectively specific strand displacement is maintained.

The probe region ensures that the molecule that the cross-linker cross-links to is the specific nucleic acid that is desired. The probe region can be a single continuous nucleic acid or it can be broken into two or more parts. In some embodiments, the probe region will include 3-50 nucleotides. In some embodiments, the probe region is less than 300 nucleotides. In some embodiments, the probe region is less than 200 nucleotides. In some embodiments, the probe region is less than 100 nucleotides. In some embodiments, the probe region is extended into the loop to further lock the probe in the open conformation once it is bound specifically to the target mRNA.

As noted above, not all cross-linking probes require activatable cross-linkers, loop regions, and/or initiator regions. In some embodiments, the cross-linking probe includes a probe region, a cross-linker, and a blocking region, that reduces the accessibility of the cross-linker to external nucleic acids. In some embodiments, the cross-linking probe further includes an initiator region. In some embodiments the initiator region is immediately adjacent and linked to the probe region such that binding of a target to the initiator region allows for strand displacement against a molecule hybridized to the probe region (e.g., the blocking region). In some embodiments, the cross-linking probe further comprises a loop region that links the probe region and the blocking region. In some embodiments, the cross-linking probe further comprises a detectable marker. In some embodiments, the cross-linking probe comprises a detectable marker region. In some embodiments, the cross-linking probe comprises a pairing region. The pairing region can be linked to the blocking region or can be linked to the probe region or loop region. In some embodiments, the pairing region can be or be part of the looped region (and thus will be accessible for hybridization upon displacement of the blocking region and the probe region.

In some embodiments, the blocking region is the same length as the probe region. In some embodiments, the blocking region is shorter than the probe region, but long enough to effectively reduce or inhibit non-target based cross-linking. In some embodiments, the blocking region comprises one or more nucleic acid strands and thus, comprises multiple subparts. In some embodiments, the blocking region has orthogonal bases. In some embodiments a part of the blocking region (e.g., the portion that might otherwise be at risk of cross-linking with the cross-linker) lacks those atoms or residues that allow for cross-linking. For example, in some embodiments, the blocking region only includes a backbone at those sections and lacks a base for cross-linking to.

As will be appreciated by one of skill in the art, the cross-linking probe and its use can be employed for any target nucleic acid, such as those nucleic acids that one may wish to observe (such as in in situ hybridization) or those that one may target for covalent modification (which would naturally impair the functionality of that nucleic acid). Exemplary target nucleic acids include, for example: RNA, DNA, mRNA, and miRNA. The target nucleic acids can be from any organism, including, but not limited to, mammals, mice, rats, primates, humans, etc. In some embodiments, the target nucleic acid remains in the host cell or subject. Thus, in some embodiments, the method can be performed in vivo, ex vivo, or in vitro. In some embodiments, the method is performed in a cell. In some embodiments, the method is performed in tissue. In some embodiments, the tissue is in a living host.

In some embodiments, the amount of the cross-linking probe provided is sufficient to allow for the determination of the presence or absence of a target nucleic acid. In some embodiments, the amount of the cross-linking probe provided is sufficient to allow for the determination of the amount of a target nucleic acid. In some embodiments, the amount of the cross-linking probe provided is sufficient to allow for binding and cross-linking to at least 1% of the target nucleic acid present in the sample to be exposed to the cross-linking probe, for example 1, 2, 3, 4, 5, 10, 15, 20, 25, 30, 35, 40, 45, 50, 55, 60, 65, 70, 75, 80, 85, 90, 95, 96, 97, 98, 99, 99.9, or greater percent of the nucleic acid target is cross-linked to a cross-linking probe, including any amount greater than any of the preceding values and any range defined between any two of the preceding values. In some applications, the concentration of probe is higher than the concentration of the target (e.g., imaging rare mRNAs). In some applications, the probe concentration may be lower (e.g., detecting DNA or RNA on a chip)

In some embodiments, one or more cross-linkers can be used in a cross-linking probe. In some embodiments, the cross-linkers are activatable. In some embodiments, the activatable cross-linker is a photo-activatable cross-linker. Exemplary photo-activatable cross-linkers include psoralens (Pieles, U. and Englisch, U. Psoralen covalently linked to oligodeoxyribonucleotides: synthesis, sequence specific recognition of DNA and photo-cross-linking to purimidine residues of DNA. Nucleic Acids Research, 1989. 17: p. 285-299), thiols (Killops, K. L., Campos, L. M., Hawker, C. J. Robust, Efficient, and Orthogonal Synthesis of Dendrimers via Thiol-ene “Click” Chemistry. Journal of the American Chemical Society, 2008. 130: p. 5062-5064), and halogenated nucleobases (Willis, M. C., et al. Photocross-linking of 5-Iodouracil-Substituted RNA and DNA to Proteins. Science, 1993. 262: p. 1255-1257) (all of which are incorporated herein by reference in their entireties and especially in regard to psoralens, thiols, and halogenated nucleobases). In some embodiments, any compound that forms interstrand cross-links when activated by ultraviolet light can be a photo-activatable cross-linker. In some embodiments, the photo-activatable cross-linker only cross-links when exposed to ultraviolet light, reducing the risk of premature cross-linking that could reduce probe sensitivity.

In some embodiments, multiple cross-linkers can be used in a single probe such that the overall yield of probes that form at least one covalent bond to the target is close to unity. In some embodiments, 1, 2, 3, 4, 5, 6, 7, 8, or more cross-linkers are used. The cross-linker(s) can be placed on either end or within the probe region. The placement of the cross-linkers within the probe region is especially advantageous. In some embodiments, where the probe region is comprised of two or more subparts, the cross-linker can be at a terminal end of one or more of the subparts.

In some embodiments, the cross-linking and cross-linker are non-promiscuous in that the cross-link is formed only between the probe region and its target and not simply to any molecule that is in the proximity of the bases that carry cross-linkers. In some embodiments, the efficiency is such that at least 80, 90, 95, 99, 99.5, 99.9, 99.99% or more of the cross-linked probe is cross-linked to a sequence that hybridizes to the probe region upon activation of the cross-linker (if activation is required). For example, in some embodiments 10, 15, 20, 25, 30, 35, 40, 45, 50, 55, 60, 65, 70, 75, 80, 85, 90, 95, 96, 97, 98, 99, 99.9, or 100% of the cross-linking probe is cross-linked to a target nucleic acid that will hybridize to the probe region, including any range greater than any of the preceding values and any range defined between any two of the preceding values. In some in vitro applications, there need be no lower limit for the above noted percentages.

In some embodiments, the cross-linker can be activated by the conformation change of the molecule. For example, in the probe region, bases carrying the cross-linker form Watson-Crick pairs between the probe region and the blocking region that are then replaced with wobble pairs to the mRNA target upon the binding of the probe region to the target. The cross-linkers have the property that they covalently link to their wobble-complement but not to their Watson-Crick complement (e.g., a G base carrying a cross-linker covalently binds to a U base but not to a C base). An example of such a cross-linker has been provided in (Coleman, R. S. and Pires, R. M. Covalent cross-linking of duplex DNA using 4-thio-2′-deoxyuridine as a readily modifiable platform for introduction of reactive functionality into oligonucleotides. Nucleic Acids Research, 1997. 25: p. 4771-4777; the entirety of which is incorporated herein by reference, including the disclosure regarding the cross-linker). In some embodiments, the cross-linker need not be activatable. Rather, the cross-linker can be one that would otherwise readily cross-link, if not for the presence of the blocking region. In such embodiments, one or more sections or subparts of the blocking region are selected so as to be inert to cross-linking (and thus avoiding the formation of initially cross-linked cross-linking probes) and the removal of the blocking region allows for the cross-linking of the cross-linker to the nucleic acid that displaces the blocking region. In some embodiments, these sections are made of orthogonal nucleotides or can simply be linkers or involve a backbone without the bases to which the cross-linkers cross-link to.

In some embodiments, the probe region and the blocking region are connected by a loop region. The loop region can include one or more nonnatural nucleotides or orthogonal nucleotides. In some embodiments, the loop region is a linker or polymer and need not be nucleotide based, as long as it can link the probe region and the blocking region. In some embodiments, the loop includes nucleotides that can bind to the target nucleic acid, thereby further enhancing the hybridization.

In some embodiments, the cross-linking probe is washed away from the sample. In some embodiments, the washing conditions are more stringent than are used for situations in which a probe is simply hybridized to a target. Thus, the washing conditions can be higher than allowed for typical in situ washes.

In some embodiments, the presence or absence of the detectable marker can be detected by imaging a sample. In some embodiments, an actual image of the sample is not required and the sample can simply be reviewed for the presence or absence of any detectable marker.

In some embodiments, the detectable marker is selected from one or more of the following: fluorescent markers (including organic fluorophores, fluorescent nucleoside analogs, and inorganic semiconductor nanocrystals), chromogenic chemical substrates or enzymes, metallic particles, organic dyes, haptens for immunochemistry, commercially available fluorophores, including quantum dots. Of course, in some embodiments, the detectable marker need not emit radiation, for example, when surface plasmon resonance is used to detect binding, the detectable marker can be the probe region itself.

In some embodiments, one or more of the herein disclosed embodiments are employed on a “DNA chip”-type application. For example, in some embodiments the probe region (which is shielded as described herein) is attached to the chip and the target is crosslinked to the chip, via the probe region only if it displaces the blocking region (which could then be washed away from the chip). In some embodiments, the probe region can be immobilized on any solid surface and then employed to pull out or detect the target nucleic acid.

Existing in situ hybridization bioimaging methods share the weakness that the background signal is raised by amplification of probes that bind non-specifically within the sample (Qian, X., L. Jin, and R. V. Lloyd, In situ hybridization: basic approaches and recent development. The Journal of Histotechnology, 2004. 27(1): p. 53-67). In some embodiments, the shielded cross-linking methods or probes can be employed to avoid or reduce this issue. One of skill in the art will also appreciate that in other embodiments, the cross-linking probes or methods do not avoid or reduce this issue.

In some embodiments, the use of the present cross-linking probes allows the detailed analysis of genetic regulatory processes.

In some embodiments, the probe and/or technique is specific, in that fluorescence signals are generated at the site of a target molecule (and in some embodiments can minimize or reduce false positives).

In some embodiments, the probe and/or technique is sensitive, in that the fluorescence signal is strong enough to enable imaging of single target molecules (and in some embodiments can minimize false negatives).

In some embodiments, the probe and/or technique is multiplexed, in that the amplification of all probe species can be performed simultaneously in parallel (and in some embodiments can enhance efficiency and reduce sample degradation).

In some embodiments, the probe and/or technique is quantitative, in that it allows quantized fluorescent signaling per target molecule (and in some embodiments can allow for relative quantification of target abundance within samples).

In some embodiments, regardless of the nature of any subsequent downstream amplification step, the present probe concept is superior to other existing probe strategies with regard to conferring specificity.

In embodiments in which the probes carry sufficiently many fluorophores of sufficient brightness and the detection device is sufficiently sensitive, then no further amplification is necessary. In some embodiments, additional amplification is desirable to increase the signal strength associated with each target molecule. In performing this amplification, it is valuable to retain the specificity that was achieved during the detection stage.

In some embodiments, the above detection and amplification schemes can be used to target multiple nucleic acid targets simultaneously by using probe and amplifier sequences that are orthogonal in sequence space. Thus, multiplexing becomes possible.

In some embodiments, the above detection and amplification schemes can be used to produce quantized fluorescent signal strength associated with each target nucleic acid that has been covalently linked to a probe molecule (and optionally to a number of additional fluorescently-labeled amplifier molecules).

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1

### mRNA Cross-Linking

This example illustrates the use of a cross-linking probe to attach a cross-linker to target mRNA.

One first obtains a cross-linking probe that includes an initiator region, a probe region that hybridizes to the desired target mRNA, a blocking region that obstructs the cross-linker when bound to the probe region, and a light activatable cross-linker. One then adds the cross-linking probe to the cell or tissue that is believed to include the target mRNA and allow the cross-linking probe to hybridize to the target mRNA. One then activates the cross-linker by irradiating the cell or tissue. Following this, the cross-linker will cross-link to the target mRNA, resulting in a cross-linked mRNA target.

### Example 2

### In Situ Hybridization

This example illustrates a use of a cross-linker probe in an in situ hybridization. One performs the steps outlined in Example 1, except that the cross-linking probe also initially includes a detectable marker region (with one or more detectable markers) that is attached to the blocking region.

Following irradiation, one then washes the tissue or cell using an ultrastringent wash containing denaturing chemicals including formamide and performing the wash at elevated temperatures. Following this, one then images the remaining detectable markers in the cell or tissue in order to identify whether or not, where, and how much of the target nucleic acid is present.

### Example 3

### mRNA In Situ Hybridization Using a Hairpin Amplifier Molecule

This example illustrates a use of a cross-linker probe in an in situ hybridization. One performs the steps outlined in Example 1, except that the cross-linking probe also initially includes a pairing region that is attached to the blocking region.

Following the irradiation, one then washes the tissue or cell via an ultrastringent wash protocol to remove a significant portion of the unbound cross-linking probe. Following this, one then adds the amplifier molecule, which includes a detectable marker region (with decteable markers) and a complementary pairing region that will bind to the pairing region on the cross-linking probe. The amplifer molecule can have the general structure depicted in FIG. 4, and the amplifier molecule can include at least one photoactivatable cross-linker in the complementary pairing region. Following this, one can then irradiate the cell or tissue a second time to cross-link the cross-linker in the amplifier molecule. Following the second irradiation, one then washes the tissue or cell to remove a significant portion of the unbound amplifier molecule.

Following this, one then images the remaining detectable markers in the cell or tissue in order to identify whether or not, where, and how much of the target nucleic acid is present.

### Example 4

### mRNA Hybridization Using a Orthogonal Base Containing Amplifier Molecule

This example illustrates a use of a cross-linker probe in an in situ hybridization. One performs the steps outlined in Example 1, except that the cross-linking probe also initially includes a pairing region that is attached to the blocking region.

Following the irradiation, one then washes the tissue or cell via an ultrastringent wash protocol to remove a significant portion of the unbound cross-linking probe. Following this, one then adds the amplifier molecule, which includes a detectable marker region (with detectable markers) and a complementary pairing region that will bind to the pairing region on the cross-linking probe and that has no consecutive natural nucleotides. The amplifer molecule can be that depicted in FIG. 5, and the amplifier molecule will include a complementary pairing region that can bind to the pairing region in the cross-linking probe. Following this, one then washes the tissue or cell to remove a significant portion of the unbound amplifier molecule.

Following this, one then images the remaining detectable markers in the cell or tissue in order to identify whether or not, where, and how much of the target nucleic acid is present.

### Example 5

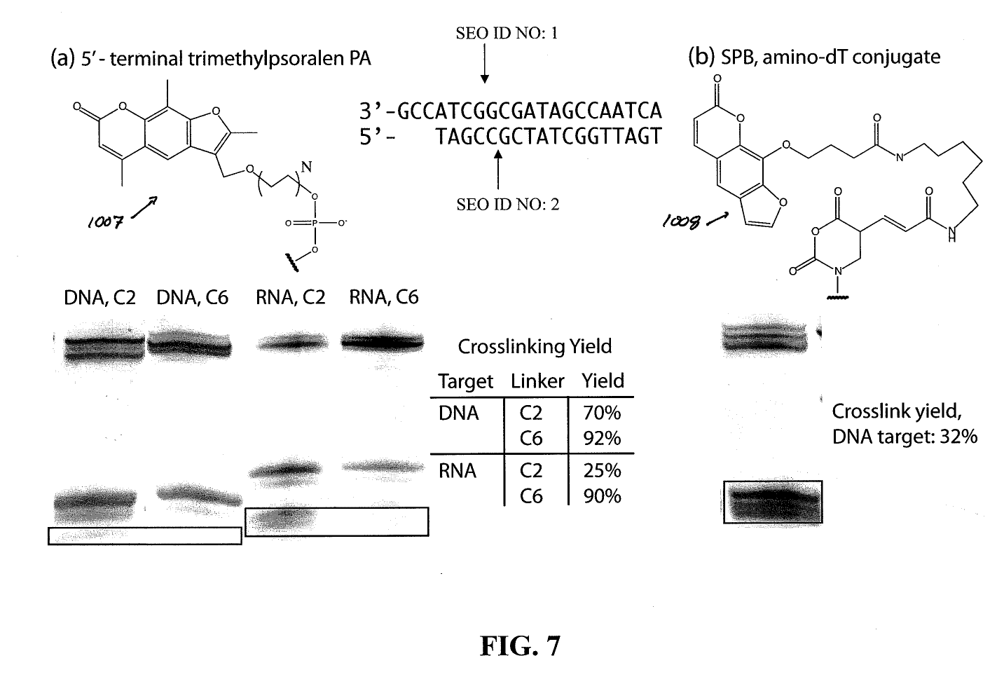

Tethered psoralens were prepared via two commercially-available products: a trimethylpsoralen (TMP) phosphoramidite 1007 (see, e.g., FIG. 7) for incorporation at the 5′-terminus of an oligonucleotide during solid-phase synthesis and a succinimidyl ester derivative 1008 (see FIG. 7) of psoralen that can be conjugated to an amine-modified oligonucleotide post-synthetically.

18 mer probes were either: (a) synthesized using 5′-terminal trimethylpsoralen phosphoramidite (Glen Research), or (b) labeled post-synthetically by conjugation of succinimidyl ester of 8-yloxy psoralen 8 (SPB, Pierce) to amine modified thymine. Probes were hybridized to 21 mer targets and irradiated with 365 m UV light for 30 min (30 mW/cm2, TMP saturated after 5 minutes, SPB 25 minutes), then analyzed by denaturing gel electrophoresis. The gels were post-stained with SyBr Gold (Invitrogen) and crosslinking yield determined by comparing the intensity of non-crosslinked target band (red box) to non-irradiated controls. The results are shown in FIG. 7.

The efficacy of both molecules in cross-linking DNA and RNA targets in vitro were examined. When attached to a probe by a 2-carbon linker, TMP cross-linked duplex DNA with moderate efficiency, but was much less effective at cross-linking to an RNA target. The results for both of these molecules are shown in FIG. 7. The 6-carbon linker enabled TMP to efficiently bind both DNA and RNA targets. The succinimidyl derivative of psoralen can be added to an oligonucleotide at any position, but the cross-linking yields were unacceptably low for DNA and lower still for RNA.

While the present teachings have been described in terms of these exemplary embodiments, the skilled artisan will readily understand that numerous variations and modifications of these exemplary embodiments are possible without undue experimentation. All such variations and modifications are within the scope of the current teachings.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein.

Unless otherwise indicated, the singular use of various words, including the term “an” or “an” denotes both the option of a single or more than one. In addition, the use of the term “and/or” denotes various embodiments that include: both options, either option in the alternative, or the combination of either option in the alternative and both options. When describing various combinations, kits, probes, methods, etc., it will be understood that unless otherwise stated, the combinations are described as comprising, consisting of, and consisting essentially of. This does not apply to the claims or to situations in the specification where the term “consisting of” is used.

### Incorporation by Reference

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

### Equivalents

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A cross-linking probe comprising:
an initiator region;
a probe region, wherein the probe region is linked to the initiator region;
at least one cross-linker that is part of the probe region; and
a blocking region that is hybridized to the probe region such that the blocking region reduces the cross-linking of the cross-linker to other molecules when the blocking region is hybridized to the probe region.

2. The cross-linking probe of claim 1, further comprising a loop region that links the probe region to the blocking region.

3. The cross-linking probe of claim 1, wherein the cross-linker is an activatable cross-linker.

4. The cross-linking probe of claim 1, wherein the activatable cross-linker is light activatable.

5. The cross-linking probe of claim 1, wherein the activatable cross-linker is conformationally activatable.

6. The cross-linking probe of claim 1, wherein the probe region comprises a first subprobe region and a second subprobe region.

7. The cross-linking probe of claim 1, wherein the probe region comprises at least on nucleotide on both sides of the cross-linker.

8. The cross-linking probe of claim 1, further comprising a detectable marker.

9. The cross-linking probe of claim 8, wherein the detectable marker is attached to a detectable marker region.

10. The cross-linking probe of claim 1, further comprising a detectable marker, wherein the detectable marker is linked to an amplifier molecule, and wherein the amplifier molecule is cross-linked to the cross-linking probe.

11. The cross-linking probe of claim 1, wherein the cross-linking probe further comprises a pairing region.

12. The cross-linking probe of claim 11, wherein the cross-linking probe is cross-linked to an amplifier molecule, wherein the amplifier molecule comprises:
a complementary pairing region that selectively hybridizes to at least a part of the pairing region, wherein the complementary pairing region comprises a cross-linker;
a blocking region that can hybridize to the complementary pairing region and can dissociate from the complementary pairing region; and
a detectable marker region linked to the complementary pairing region, wherein the detectable marker region comprises one or more detectable markers.

13. The cross-linking probe of claim 1, wherein the probe further comprises a pairing region.

14. The cross-linking probe of claim 13, where the cross-linking probe is cross-linked to an amplifier molecule, wherein the amplifier molecule comprises:
a complementary pairing region that selectively hybridizes to the pairing region; and
a detectable marker region, comprising one or more detectable markers.

15. The cross-linking probe of claim 13, wherein the pairing region comprises one or more orthogonal nucleotides.

16. The cross-linking probe of claim 13, wherein there are no consecutive natural bases in the pairing region.

17. The cross-linking probe of claim 1, wherein the cross-linker comprises an extender linker.

18. The cross-linking probe of claim 1, wherein the probe region is immediately adjacent to the initiator region.

19. The cross-linking probe of claim 1, further comprising detectable marker region that comprises at least one fluorescent label.

20. The cross-linking probe of claim 1, further comprising a first monomer that is cross-linked to the probe region.

21. The cross-linking probe of claim 20, further comprising a second monomer crosslinked to the first monomer.

22. A method of associating a cross-linking probe with a nucleic acid sequence, said method comprising:
providing a cross-linking probe and a nucleic acid sequence; wherein the cross-linking probe comprises:
an initiator region;
a probe region, wherein the probe region is linked to the initiator region;
at least one cross-linker that is part of the probe region; and
a blocking region hybridized to the probe region;
hybridizing the initiator region to a part of the nucleic acid sequence;
dissociating the blocking region from the probe region;
hybridizing the probe region to a second part of the nucleic acid sequence; and
cross-linking the cross-linker.

23. The method of claim 22, further comprising performing an ultrastringent wash following cross-linking.

24. The method of claim 22, wherein the cross-linking probe further comprises a detectable marker.

25. The method of claim 24, further comprising the step of detecting the presence or absence of the detectable marker following the ultrastringent wash.

26. The method of claim 25, wherein the detectable marker is attached to a detectable marker region.

27. The method of claim 26, wherein the detectable marker region is linked to the blocking region.

28. The method of claim 22, wherein the cross-linking probe is associated with a detectable marker region, wherein the detectable marker region is part of an amplifier molecule, and wherein the amplifier molecule is cross-linked to the cross-linking probe.

29. The method of claim 22, wherein the cross-linking probe is associated with a detectable marker region, wherein the detectable marker region is part of an amplifier molecule, and wherein the amplifier molecule is hybridized to the cross-linking probe, wherein the amplifier molecule comprises one or more orthogonal nucleotides.

30. The method of claim 22, wherein the method is performed in a cell.

31. The method of claim 22, wherein the method is performed in vitro.

32. The method of claim 22, wherein the method is performed in vivo.

33. A method of determining the presence or absence of a nucleic acid, said method comprising:
providing a sample;
adding to the sample an initiator region that is linked to a probe region, wherein there is at least one uncross-linked cross-linker that is part of the probe region, and wherein a blocking region is hybridized to the probe region when the initiator region is added to the sample;
hybridizing the initiator region to a first part of a nucleic acid contained within the sample, if the nucleic acid is present in the sample;
dissociating the blocking region from the probe region, if the nucleic acid is present in the sample;
hybridizing the probe region to a second part of the nucleic acid, if the nucleic acid is present in the sample;
cross-linking the cross-linker;
performing a wash following the cross-linking;
associating the probe region with a detectable marker; and
detecting the presence or absence of the detectable marker, thereby determining the presence or absence of a nucleic acid.

34. The method of claim 33, wherein the detectable marker is linked to a detectable marker region that is linked to the blocking region.

35. The method of claim 33, wherein the probe region is linked to a pairing region, and wherein the detectable marker is attached to a first monomer that is crosslinked to the pairing region.

36. The method of claim 35, further comprising a second monomer that is crosslinked to the first monomer.

37. The method of claim 33, wherein the detectable marker is attached to an amplifier molecule that comprises a pairing region that hybridizes to a complementary pairing region that is linked to the initiator region, wherein the pairing region comprises at least a second cross-linker.

38. The method of claim 37, wherein the amplifier molecule is added after the wash, and wherein following the addition of the amplifier molecule, the second cross-linker is cross-linked and a second wash is performed.

39. The method of claim 37, wherein the detectable marker is attached to an amplifier molecule that comprises a pairing region that hybridizes to a complementary pairing region that is linked to the initiator region, wherein the pairing region comprises at least one orthogonal nucleotide.

40. The method of claim 37, wherein the initiator region and the probe region are no longer than 50 nucleotides in length.

41. The method of claim 37, wherein the cross-linking probe is no longer than 1000 nucleotides in length.

42. The cross-linking probe of claim 4, wherein the activatable cross-linker comprises psoralen.


SEQUENCE LISTING (2 sequences)

SEQ ID NO: 1 - DNA, 21 bp, Artificial Sequence
  Primer
actaaccgatagcggctaccg

SEQ ID NO: 2 - DNA, 18 bp, Artificial Sequence
  Primer
tagccgctatcggttagt